(19) United States
(12) Patent Application Publication
(10) Pub. No.: US 2025/0014766 A1
(43) Pub. Date: Jan. 9, 2025

(54) DRUG REACTION PREDICTION AND MODEL TRAINING METHOD, APPARATUS AND DEVICE

(71) Applicant: BEIJING BAIDU NETCOM SCIENCE TECHNOLOGY CO., LTD., Beijing (CN)

(72) Inventors: Jingbo ZHOU, Beijing (CN); Yuhan YE, Beijing (CN)

(73) Assignee: BEIJING BAIDU NETCOM SCIENCE TECHNOLOGY CO., LTD., Beijing (CN)

(21) Appl. No.: 18/895,554

(22) Filed: Sep. 25, 2024

(30) Foreign Application Priority Data
Jun. 19, 2024 (CN) 202410796892.4

(51) Int. Cl. G16H 70/40; G06N 20/00

(52) U.S. Cl. CPC G16H 70/40; G06N 20/00

(57) ABSTRACT

A drug reaction prediction method, which is related to the field of artificial intelligence, specifically involving deep learning, computational biology, and chemistry, is disclosed. The drug reaction prediction method includes: obtaining a target graph based on multiple levels of entities contained in a drug to be predicted; the target graph includes an entity graph representing topological information within the entities and an interaction graph representing correlation information between the entities; performing representation extraction processing on the target graph to obtain an initial representation; obtaining a target representation based on a predetermined prompt identifier and the initial representation; and obtaining a drug reaction prediction result for the drug to be predicted based on the target representation.

### CROSS-REFERENCE TO RELATED APPLICATION

The present application claims the priority and benefit of Chinese Patent Application No. 202410796892.4, filed on Jun. 19, 2024, entitled “DRUG REACTION PREDICTION AND MODEL TRAINING METHOD, APPARATUS AND DEVICE”. The disclosure of the above application is incorporated herein by reference in its entirety.

### TECHNICAL FIELD

The present disclosure relates to the field of artificial intelligence, specifically to deep learning, computational biology and chemistry, and more particularly to drug reaction prediction and model training method, apparatus and device.

### BACKGROUND

In the fields of computational biology and computational chemistry, accurately predicting various side effects of drug molecules is crucial for enhancing drug efficacy and other applications, such as combination therapy and clinical decision-making.

The challenge lies in how to precisely predict drug reactions.

### SUMMARY

The present disclosure provides drug reaction prediction method, electronic device and storage medium.

According to one aspect of the present disclosure, a drug reaction prediction method is provided, which includes: obtaining a target graph based on multiple levels of entities contained in a drug to be predicted; the target graph includes an entity graph representing topological information within the entities and an interaction graph representing correlation information between the entities; performing representation extraction processing on the target graph to obtain an initial representation; obtaining a target representation based on a predetermined prompt identifier and the initial representation; and obtaining a drug reaction prediction result for the drug to be predicted based on the target representation.

According to another aspect of the present disclosure, an electronic device is provided, which includes: at least one processor; and a memory communicatively connected to the at least one processor; where the memory stores instructions executable by the at least one processor, the instructions, when executed by the at least one processor, enabling the at least one processor to perform the method according to any one of the aforementioned aspects.

According to another aspect of the present disclosure, a non-transitory computer-readable storage medium is provided, storing computer instructions, where the computer instructions are configured to cause the computer to perform the method according to any one of the aforementioned aspects.

It should be understood that the contents of this section are not intended to identify key or essential features of the embodiments of the present disclosure, nor are they intended to limit the scope of the present disclosure. Other features of the present disclosure will become apparent from the following description.

### BRIEF DESCRIPTION OF THE DRAWINGS

The drawings are provided to better understand the present disclosure and are not intended to limit the scope of the present disclosure.

FIG. 1 is a schematic diagram according to a first embodiment of the present disclosure.

FIG. 2 is a schematic diagram of an application scenario for implementing the drug reaction prediction method according to an embodiment of the present disclosure.

FIG. 3 is a structural schematic diagram of the drug reaction prediction model provided by an embodiment of the present disclosure.

FIG. 4 is a schematic diagram according to a second embodiment of the present disclosure.

FIG. 5 is a schematic diagram according to a third embodiment of the present disclosure.

FIG. 6 is a schematic diagram according to a fourth embodiment of the present disclosure.

FIG. 7 is a schematic diagram according to a fifth embodiment of the present disclosure.

FIG. 8 is a schematic diagram of an electronic device for implementing the drug reaction prediction method or the drug reaction prediction model training method according to embodiments of the present disclosure.

### DETAILED DESCRIPTION OF EMBODIMENTS

The following description, including the drawings, provides explanations of exemplary embodiments of the present disclosure, which include various details to aid understanding and should be considered merely as illustrative. Therefore, it should be recognized by those of ordinary skill in the art that various changes and modifications can be made to the described embodiments without departing from the scope and spirit of the present disclosure. For clarity and conciseness, descriptions of well-known functions and structures have been omitted in the following description.

Existing drug reaction prediction methods in the related art suffer from insufficient accuracy.

To improve the accuracy of drug reaction prediction, the present disclosure provides the following embodiments.

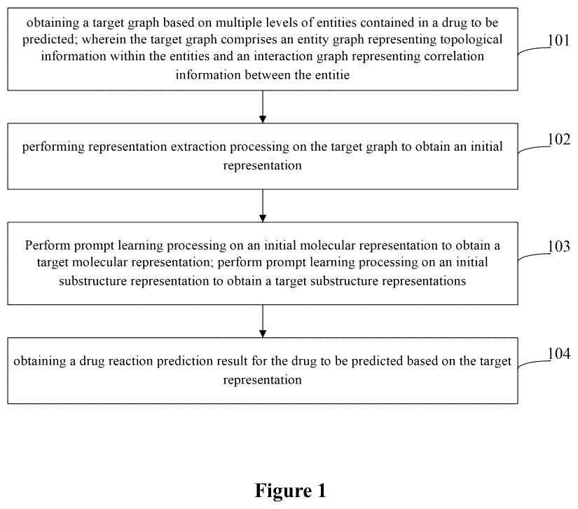

FIG. 1 is a schematic diagram according to a first embodiment of the present disclosure. This embodiment provides a drug reaction prediction method, which includes:

101. Obtain a target graph based on multiple levels of entities contained in a drug to be predicted; the target graph includes an entity graph representing topological information within the entities, and an interaction graph representing correlation information between the entities.

102. Perform representation extraction processing on the target graph to obtain an initial representation.

103. Perform prompt learning processing on an initial molecular representation to obtain a target molecular representation; perform prompt learning processing on an initial substructure representation to obtain a target substructure representations.

104. Obtain a drug reaction prediction result for the drug to be predicted based on the target representation.

A drug is composed of drug molecules, which can be regarded as a molecular graph structure with various types of atoms as nodes and chemical bonds as edges, containing rich topological structure information. Each drug molecule includes one or more substructures.

To this end, entities include the aforementioned drug molecules. To enhance prediction accuracy, entities at other levels are also considered. For example, at least one target substructure of the drug molecule is also included.

The above takes the multiple levels of entities including a drug molecule and a target substructure as an example. However, it should be understood that other number of levels can also be included, such as different levels of substructures. Specifically, the molecular structure corresponding to the drug molecule can be divided into multiple levels of substructures, which are then treated as the multiple levels of entities.

The target substructure refers to an important substructure of the drug molecule, which can be extracted through a molecular cleavage process.

The specific extraction process may include: first, preprocessing the complex molecule and cleaving the corresponding ligands; second, applying a decomposition method based on chemical domain knowledge to cut the molecule into fragments; finally, combining overly fragmented segments based on certain post-processing rules, and screening out excessively rare substructures based on their frequency, thereby obtaining effective important substructure(s) as the target substructure(s).

Taking the multiple levels of entities including a drug molecule and its target substructure as an example, the generated entity graph can be referred to as a molecular graph and a substructure graph, respectively; the interaction graph specifically refers to the interaction graph between the drug molecule and the target substructure.

The molecular graph refers to the structural graph corresponding to the chemical formula of the drug molecule, which is uniquely generated based on preset rules. For example, a molecule is composed of various types of atoms, with each type of atom considered as a node and a chemical bond between atoms considered as an edge.

The substructure graph is the important substructure within the structural graph corresponding to the molecule, where its nodes are some atoms within the molecule and the edges are the chemical bonds between these atoms.

The aforementioned entity graph can represent topological information within the corresponding entities, such as the molecular graph representing internal topological information of the molecule and the substructure graph representing internal topological information of the substructure.

The interaction graph refers to a graph generated based on the drug molecule and the target substructure, used to represent the correlation information between different entities within the drug, where the entities include the drug molecule and the target substructure.

The nodes of the interaction graph include the nodes corresponding to the drug molecule and the nodes corresponding to the target substructure, and the edges include: an edge between nodes corresponding to drug molecules, which represent the similarity relationship between the drug molecules; an edge between nodes corresponding to target substructures, which represent the similarity relationship between the target substructures; and an edges between a node corresponding to a drug molecule and a node corresponding to a target substructure, which indicate the inclusion relationship between the drug molecule and the target substructure.

Assuming the number of drug molecules is D and the number of target substructures is M, there are a total of (D+M) entity graphs, and the number of nodes in the interaction graph is (D+M). Both D and M are positive integers.

After obtaining the target graph, representation extraction processing is performed on the target graph to obtain an initial representation. Since the target graph is a graph structure, a graph neural network can be used for representation extraction processing.

After obtaining the initial representation, prompt learning processing can be performed on the initial representation based on a predetermined prompt identifier to obtain a target representation.

After obtaining the target representation, a prediction can be made based on the target representation to obtain a drug reaction prediction result.

The drug reaction prediction result can specifically be a drug reaction category.

Prompt learning refers to guiding the reasoning process of a model through prompting.

In the field of natural language processing, for a specific downstream task, providing a textual prompt can enable a pre-trained language model to directly apply to that downstream task.

In this embodiment, since the processing is performed on graphs, it can be referred to as graph prompt learning, which primarily involves obtaining a target representation based on a prompt identifier and the initial representation, and using the target representation as new inputs for drug reaction prediction. The prompt identifier can be specifically determined through a training process.

In this embodiment, obtaining a representation based on the target graph, since the target graph includes an entity graph and an interaction graph, which represent the internal topological information of entities and the correlation information between entities, respectively, this allows for predictions based on both internal and external information, thereby improving the accuracy of drug reaction prediction; additionally, obtaining target representations based on prompt identifiers and initial representations enables prompt learning on the representations, facilitating the effective transfer of pre-trained knowledge and further enhancing prediction accuracy.

To better understand the embodiments of the present disclosure, an explanation of the applicable application scenarios is provided.

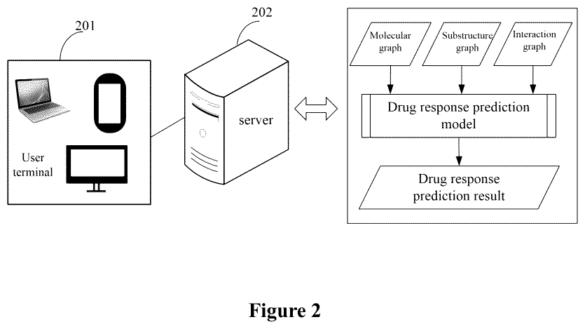

FIG. 2 is a schematic diagram of the application scenario for implementing the drug reaction prediction method according to an embodiment of the present disclosure. The drug reaction prediction process can be implemented through rules or deep learning network models, for example, this embodiment uses a deep learning network model. This model can be referred to as a drug reaction prediction model.

As shown in FIG. 2, a user terminal 201 may install an application (APP) for drug reaction prediction. This APP interacts with a server-side, which is deployed on a server 202, and can be a local server or a cloud server, and can be a single server or a server cluster. The user terminal can connect to the server via a wired or wireless network.

A user can send relevant information about the drug to be predicted, such as the drug name or identification information, or the chemical formula or molecular structure diagram of the drug molecule, to the server-side via the APP. The server-side constructs entity graphs (molecular graphs and substructure graphs) and interaction graphs between the drug molecule and the target substructure based on this information. For example, the server-side obtains the molecular graph of the drug molecule to be predicted according to pre-configured information or queries, and then acquires the target substructure and its corresponding substructure graph based on the molecular graph, and constructs the interaction graph according to the molecule and the target substructure. Subsequently, the drug reaction prediction model is used to process the input entity graphs and interaction graphs, and the output is the drug reaction prediction result, which can specifically be the drug reaction category. The server-side can then feed back this prediction result to the APP, which displays it to the user. This embodiment uses server-side processing as an example; it should be understood that if the user terminal itself has the corresponding capabilities, the relevant processes can also be executed locally on the user terminal.

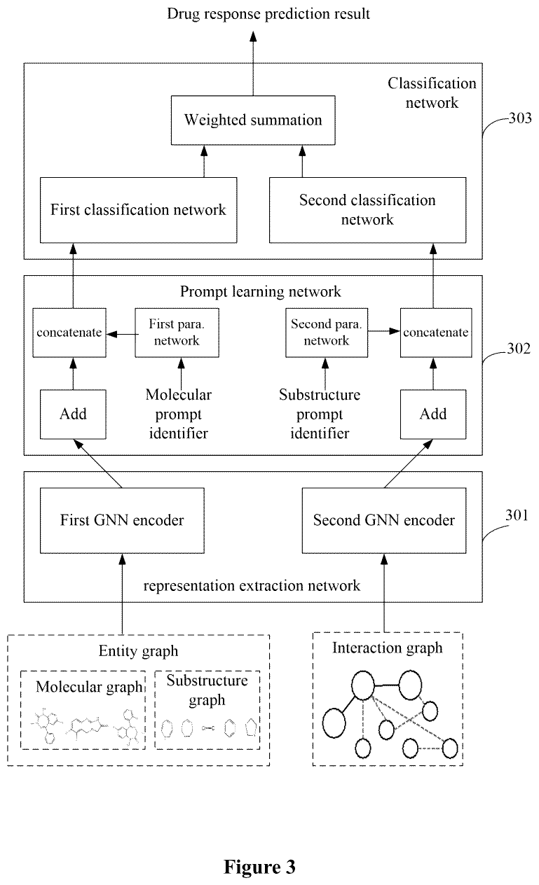

FIG. 3 is a schematic diagram of the drug reaction prediction model provided by an embodiment of the present disclosure.

As shown in FIG. 3, the drug reaction prediction model includes: a representation extraction network 301, a prompt learning network 302, and a classification network 303.

The input to the representation extraction network is the target graph, which includes an entity graph and an interaction graph; the entity graph includes a molecular graph and a substructure graph, and the interaction graph is constructed based on the drug molecule and its target substructure; the output is the representation, with the representation corresponding to the entity graph referred to as an initial molecular representation, and the representation corresponding to the interaction graph referred to as an initial substructure representation.

The input to the prompt learning network includes the initial representation and a prompt identifier, and the output is a target representation. During the training phase, the prompt identifier is learnable, i.e., adjustable through a loss function; during the prediction phase, the final trained prompt identifier is used.

The input to the classification network is the target representation, and the output is the drug reaction prediction result.

Further, the representation extraction network may include: a first target encoder and a second target encoder, which respectively obtain the initial molecular representation and the initial substructure representation. These target encoders can be pre-trained models.

Specifically, the aforementioned encoders can be Graph Neural Network (GNN) encoders, referred to as a first GNN encoder and a second GNN encoder.

The input to the first GNN encoder is the entity graph, including the molecular graph and the substructure graph, and the output is the initial molecular representation; the input to the second GNN encoder is the interaction graph, and the output is the initial substructure graph representation.

The prompt learning network may include: a first parameterization network and a second parameterization network; the input to the first parameterization network is the molecular prompt identifier, and the output is the molecular prompt representation; the input to the second parameterization network is the substructure prompt identifier, and the output is the substructure prompt representation. Other operations, such as addition and concatenation, can also be included in the prompt learning network, after which the target molecular representation and the target substructure representation are obtained.

The classification network may include: a first classification network and a second classification network; the input to the first classification network is the target molecular representation, and the output is the first prediction result; the input to the second classification network is the target substructure representation, and the output is the second prediction result. Subsequently, weighted summation may be performed on the first prediction result and the second prediction result to obtain the final prediction result, i.e., the drug reaction prediction result.

In conjunction with the aforementioned application scenario, the present disclosure also provides the following embodiments.

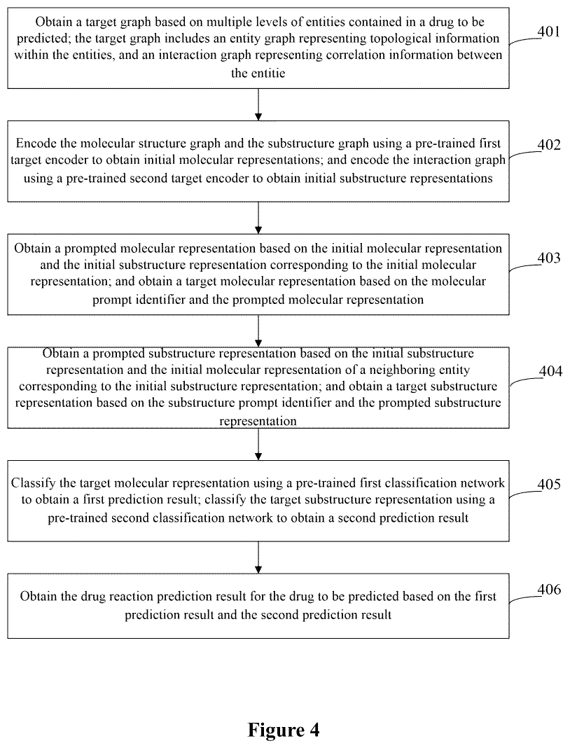

FIG. 4 is a schematic diagram according to a second embodiment of the present disclosure. This embodiment provides a drug reaction prediction method, which includes:

401. Obtain a target graph based on multiple levels of entities contained in a drug to be predicted; the target graph includes an entity graph representing topological information within the entities and an interaction graph representing correlation information between the entities.

Among these, the multiple levels of entities include: one or more drug molecules and respective target substructures of the one or more drug molecules; correspondingly, the entity graph include: one or more molecular structure graphs corresponding respectively to the drug molecules, and the substructure graphs corresponding respectively to the target substructures.

402. Encode the molecular structure graph and the substructure graph using a pre-trained first target encoder to obtain initial molecular representations; and encode the interaction graph using a pre-trained second target encoder to obtain initial substructure representations.

In this embodiment, by using two target encoders, initial molecular representations and initial substructure representations can be obtained separately, which can leverage the excellent performance of the encoders to improve the accuracy of the initial representations, thereby enhancing prediction accuracy.

403. Obtain a prompted molecular representation based on the initial molecular representation and the initial substructure representation corresponding to the initial molecular representation; and obtain a target molecular representation based on the molecular prompt identifier and the prompted molecular representation.

404. Obtain a prompted substructure representation based on the initial substructure representation and the initial molecular representation of a neighboring entity corresponding to the initial substructure representation; and obtain a target substructure representation based on the substructure prompt identifier and the prompted substructure representation.

In this embodiment, processing the initial representation based on prompt identifier to obtain target representation can align the downstream objectives with the pre-training tasks using prompt learning, facilitating knowledge transfer and improving prediction accuracy.

Further, add the initial molecular representation and the initial substructure representation corresponding to the initial molecular representation to obtain the prompted molecular representation; and/or add the initial substructure representation and the initial molecular representation of a neighboring entity corresponding to the initial substructure representation to obtain the prompted substructure representation.

Specifically, this can be expressed by the following formulas:

$${\tilde{z}}_{i} = {f}_{p}^{m} ( h \vert {h}_{i}^{D} + {h}_{i}^{M} , {v}_{i}^{D} ∈ V )$$

$${\overline{z}}_{j} = {f}_{p}^{m} ( h \vert {h}_{j}^{D} + {h}_{j}^{M} , {v}_{j}^{M} ∈ V , {v}_{j}^{D} ∈ N ( {v}_{i}^{D} ) )$$

Where, \tilde{z}ᵢ, z̅ⱼ represents the prompted molecular representation and the prompted substructure representation respectively; hₜ^{D}, hⱼ^{D} represents the initial molecular representations; hᵢ^{M}, hₖ^{M} represents the initial substructure representations; vᵢ^{D} is the i-th entity; vⱼ^{M} is the j-th node of the interaction graph, with i and j being quantity indices; V is the union set of the drug molecule set and the target substructure set; N(vᵢ^{D}) is the neighboring entity of vᵢ^{D}.

In this embodiment, by adding the initial molecular representation and the initial substructure representation to obtain the prompted representation, information from both the drug molecule and substructure perspectives can be integrated, enhancing the expressive power of the prompted representation and thereby improving the accuracy of drug reaction prediction.

Further, parameterize the molecular prompt identifier using a pre-trained first parameterization network to obtain a molecular prompt representation; concatenate the molecular prompt representation with the prompted molecular representation to obtain the target molecular representation; and/or parameterize the substructure prompt identifier using a pre-trained second parameterization network to obtain substructure prompt representation; concatenate the substructure prompt representation with the prompted substructure representation to obtain the target substructure representation.

Specifically, this can be expressed by the following formulas:

$${P}_{d}^{′} = {Θ}_{d} ( {P}_{d} ) , {P}_{m}^{′} = {Θ}_{m} ( {P}_{m} )$$

$${\tilde{z}}^{′} = {f}_{p}^{a} ( \tilde{z} ) = [ {P}_{d}^{′} ⊕ \tilde{z} ] , {\tilde{z}}^{′} = {f}_{p}^{a} ( \overline{z} ) = [ {P}_{m}^{′} ⊕ \overline{z} ]$$

Where \tilde{z}′, z̅′ are the target molecular representation and target substructure representation, respectively; Θ_{d}, Θₘ are the first parameterization network and second parameterization network, respectively; P_{d}, Pₘ are the molecular prompt identifier and the substructure prompt identifier, respectively; P′_{d}, P′ₘ are the molecular prompt representation and substructure prompt representation, respectively; \tilde{z}, z̅ are the prompted molecular representation and prompted substructure representation, respectively; ⊕ denotes the concatenation operation.

In this embodiment, by concatenating the prompt representation and the prompted representation to obtain the target representation, prompt information can be embedded in the target representation, facilitating better prediction and improving prediction performance.

405. Classify the target molecular representation using a pre-trained first classification network to obtain a first prediction result; classify the target substructure representation using a pre-trained second classification network to obtain a second prediction result.

406. Obtain the drug reaction prediction result for the drug to be predicted based on the first prediction result and the second prediction result.

In this embodiment, obtaining the final prediction result based on the first and second prediction results allows for the integration of prediction information from both the drug molecule perspective and the drug substructure perspective, thereby improving the accuracy of the final prediction result.

Further, a pre-trained linear projection layer can be used to process the target molecular representation and the target substructure representation to obtain weights; based on these weights, weighted summation may be performed on the first prediction result and the second prediction result to obtain the drug reaction result.

Specifically, this can be expressed by the following formulas:

$${\tilde{y}}_{p r e d} = p ( {\tilde{z}}^{′} , r ) , {\overline{y}}_{p r e d} = p ( {\overline{z}}^{′} , r )$$

$$η = sigmoid ( {W}_{s} ( {\tilde{z}}^{′} , {\overline{z}}^{′} ) )$$

$${y}_{pred} = η {\tilde{y}}_{pred} + ( 1 - η ) {\overline{y}}_{pred}$$

Where y_{prod} is the final prediction result, i.e., the drug reaction prediction result; \tilde{y}_{pred}, y̅_{pred} are the first prediction result and the second prediction result, respectively, p represents probability, which is the output of the two classification networks, r represents the reaction category; η is the weight; Wₛ is the linear projection layer; \tilde{z}′, z̅′ are the target molecular representation and target substructure representation, respectively.

In this embodiment, by determining the weights and performing a weighted summation on the two prediction results based on these weights, it is possible to more accurately integrate the prediction information from both the drug molecule and the drug substructure, thereby improving the accuracy of the drug reaction prediction result.

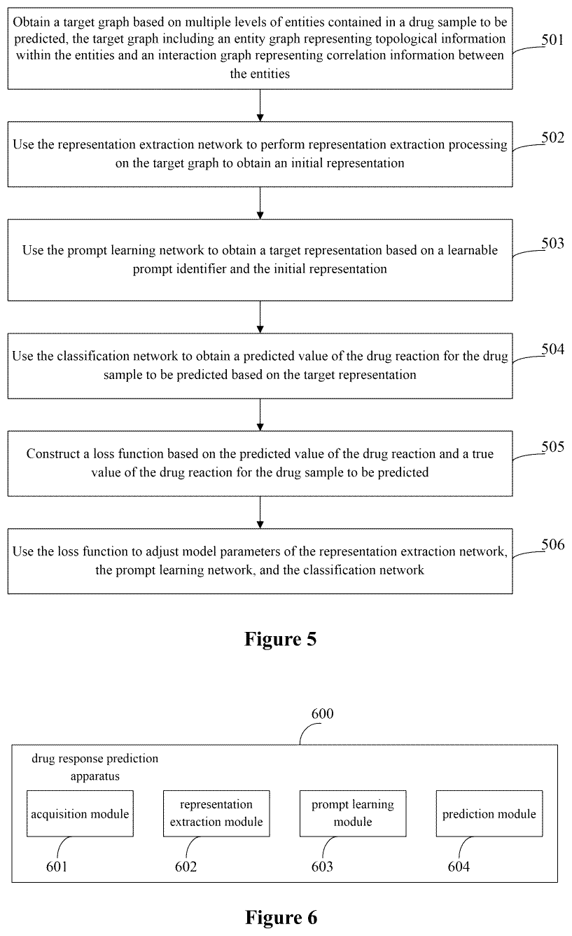

FIG. 5 is a schematic diagram according to a third embodiment of the present disclosure. This embodiment provides a method for training a drug reaction prediction model, which includes a representation extraction network, a prompt learning network, and a classification network. The training method includes:

501. Obtain a target graph based on multiple levels of entities contained in a drug sample to be predicted, the target graph including an entity graph representing topological information within the entities and an interaction graph representing correlation information between the entities.

502. Use the representation extraction network to perform representation extraction processing on the target graph to obtain an initial representation.

503. Use the prompt learning network to obtain a target representation based on a learnable prompt identifier and the initial representation.

504. Use the classification network to obtain a predicted value of the drug reaction for the drug sample to be predicted based on the target representation.

505. Construct a loss function based on the predicted value of the drug reaction and a true value of the drug reaction for the drug sample to be predicted.

506. Use the loss function to adjust model parameters of the representation extraction network, the prompt learning network, and the classification network.

The drug sample to be predicted and its corresponding true value of drug reaction are obtained in advance, such as from an existing dataset, or obtained through manual annotation or other annotation methods.

Using a similar process to the prediction process, during the training phase, the output of the classification network is referred to as the drug reaction prediction value. Subsequently, based on this output drug reaction prediction value and the corresponding true drug reaction value, a loss function is constructed. Then, the model parameters are adjusted based on the loss function, such as adjusting the model's weight coefficients w and bias coefficients b, until a preset termination condition is met, and the model that meets the preset termination condition is used as the final model.

The expression of the aforementioned loss function can be as follows:

$$L = \sum I ( {y}_{p r e d} \vert {y}_{t r u e} )$$

Where L is the loss function; yₚᵣₑ, is the drug reaction prediction value; yₜᵣᵤₑ is the true drug reaction value; I is the expression of the loss function, such as the cross-entropy loss function.

Additionally, the prompt identifier is learnable, meaning that the aforementioned loss function can also be used to adjust the prompt identifier, with the adjustment method similar to that of the model parameters.

In this embodiment, obtaining representations based on the target graph, since the target graph includes entity graphs and interaction graphs, which represent the internal topological information of entities and the correlation information between entities, respectively, this allows for training based on both internal and external information, improving the model's effectiveness and thus enhancing the accuracy of drug reaction prediction. Furthermore, obtaining target representations based on prompt identifiers and initial representations enables prompt learning on the representations, facilitating the effective transfer of pre-trained knowledge and further enhancing the model's and prediction accuracy.
In some embodiments, the multiple levels of entities include: a drug molecule and a target substructure of the drug molecule;
The entity graph includes: the molecular structure graph corresponding to the drug molecule, and the substructure graph corresponding to the target substructure;
The representation extraction network includes: a first target encoder and a second target encoder;

The step of using the representation extraction network to perform representation extraction processing on the target graph to obtain initial representations includes at least one of the following:
Encoding the molecular structure graph and the substructure graph using the first target encoder to obtain an initial molecular representation;
Encoding the interaction graph using the second target encoder to obtain an initial substructure representation;

The initial representation include: an initial molecular representation and an initial substructure representation.

In this embodiment, by using two target encoders, initial molecular representation and initial substructure representation can be obtained separately, which can leverage the excellent performance of the encoders to improve the accuracy of the initial representation, thereby enhancing prediction accuracy.
In some embodiments, the prompt identifier include: a molecular prompt identifier and a substructure prompt identifier;
The prompt learning network includes: a first parameterization network and a second parameterization network;

The step of using the prompt learning network to obtain the target representation based on learnable prompt identifier and the initial representation includes at least one of the following:
Obtaining a prompted molecular representation based on the initial molecular representation and the initial substructure representation corresponding to the initial molecular representation; using the first parameterization network to parameterize the molecular prompt identifier to obtain a molecular prompt representation; and obtaining a target molecular representation based on the molecular prompt representation and the prompted molecular representation;
Obtaining a prompted substructure representation based on the initial substructure representation and the initial molecular representation of a neighboring entity corresponding to the initial substructure representation; using the second parameterization network to parameterize a substructure prompt identifier to obtain a substructure prompt representation; and obtaining the target substructure representation based on the substructure prompt representation and the prompted substructure representation;

The target representation include: the target molecular representation and the target substructure representation.

In this embodiment, processing the initial representation based on the prompt identifier to obtain a target representation can use prompt learning to align downstream objectives with pre-training tasks, facilitating knowledge transfer and improving prediction accuracy.
In some embodiments, the target representation include: target molecular representation and target substructure representation;
The classification network includes: a first classification network and a second classification network;

The step of using the classification network to obtain the predicted value of the drug reaction for the drug sample to be predicted based on the target representation includes:
Using the first classification network to classify the target molecular representation to obtain a first prediction result;
Using the second classification network to classify the target substructure representations to obtain a second prediction result;
Obtaining the drug reaction prediction value based on the first prediction result and the second prediction result.

In this embodiment, obtaining the final prediction result based on the first and second prediction results allows for the integration of prediction information from both the drug molecule perspective and the drug substructure perspective, thereby improving the accuracy of the final prediction result.

The aforementioned training process can specifically be a fine-tuning process, where the first target encoder and the second target encoder can be pre-trained encoders, and the pre-trained encoders and other related networks are fine-tuned through the aforementioned training process to obtain the final drug reaction prediction model.

The final drug reaction prediction model can be used for the drug reaction prediction process in any of the aforementioned embodiments, and due to the high accuracy of the drug reaction prediction model, the accuracy of drug reaction prediction can be improved.

To this end, in addition to the aforementioned fine-tuning stage, the training phase may also include a pre-training stage.

Accordingly, the training method may also include:
Obtaining a pre-trained molecular structure graph, a pre-trained substructure graph, and a pre-trained interaction graph based on a pre-training sample;
Encoding the pre-trained molecular structure graph and the pre-trained substructure graph using a first encoder to obtain a pre-trained molecular representation;
Encoding the pre-trained interaction graph using a second encoder to obtain a pre-trained substructure representation;
Constructing a pre-training loss function based on the pre-trained molecular representation and the pre-trained substructure representation;
Adjusting the model parameters of the first encoder and the second encoder based on the pre-training loss function until a pre-training termination condition is met;
Using the first encoder and the second encoder that meet the pre-training termination condition as the first target encoder and the second target encoder, respectively.

In this embodiment, the pre-training process may be used to obtain the first target encoder and the second target encoder, which allows for further fine-tuning on the pre-training basis to obtain the final drug reaction prediction model, thereby reducing workload and improving training efficiency and generalization.

In some embodiments, constructing the pre-training loss function based on the pre-trained molecular representation and the pre-trained substructure representation includes:
Constructing a molecular level loss function based on the pre-trained molecular representation;
Constructing a substructure level loss function based on the pre-trained substructure representation;
Constructing a contrastive loss function based on the pre-trained molecular representation and the pre-trained substructure representation;
Constructing the pre-training loss function based on the molecular level loss function, the substructure level loss function, and the contrastive loss function.

This can be expressed by the following formulas:

$${L}_{drug} = \sum {l}_{drug} ( {F}^{s i m} ( {h}_{i}^{D} , {h}_{j}^{D} ) ; {y}_{s i m} )$$

$${L}_{motif} = \sum {l}_{m o t i f} ( {F}^{c o n} ( {h}_{i}^{M} , {h}_{j}^{M} ) ; {y}_{c o n} )$$

$${L}_{pre} = {L}_{drug} + {L}_{motif} + {L}_{cl}$$

Where Lₚᵣₑ is the pre-training loss function; L_{drug} is the molecular level loss function; L_{motif} is the substructure level loss function; L_{cl} is the contrastive loss function, which is constructed based on positive and negative samples, where the pre-trained molecular representation and pre-trained substructure representation corresponding to the same drug molecule are positive samples, and the rest are negative samples, with specific formulas referring to the contrastive learning loss function formulas in related technologies; hᵢ^{D}, hⱼ^{D} are any two pre-trained molecular representations; hᵢ^{M}, hⱼ^{M} are any two pre-trained substructure representations; F^{sim}( ) is the similarity function, such as cosine similarity or a fully connected layer; yₛᵢₘ is the similarity as the learning target, which can be calculated by the molecular fingerprints between drug molecules; l_{drug} is the loss function at the drug molecule level, such as the mean squared error function; F^{con}( ) is the reconstruction network, such as a fully connected network, where the input to the reconstruction network is the graph with edges randomly masked in the interaction graph, and the output is the reconstructed information of the randomly masked edges; y_{con} is the connectivity information as the learning target, used to indicate whether an edge exists.

In this embodiment, constructing the pre-training loss function based on the molecular level loss function, the substructure level loss function, and the contrastive loss function can combine multiple types of information to build the pre-training loss function, improving the pre-training effect and thus enhancing the model and prediction performance.

FIG. 6 is a schematic diagram according to a fourth embodiment of the present disclosure. This embodiment provides a drug reaction prediction apparatus, denoted as apparatus 600, which includes: an acquisition module 601, a representation extraction module 602, a prompt learning module 603, and a prediction module 604.

The acquisition module 601 is configured to obtain a target graph based on multiple levels of entities contained in a drug to be predicted; the target graph includes an entity graph representing topological information within the entities and an interaction graph representing correlation information between the entities; the representation extraction module 602 is configured to perform representation extraction processing on the target graph to obtain an initial representation; the prompt learning module 603 is configured to obtain a target representation based on a predetermined prompt identifier and the initial representation; the prediction module 604 is configured to obtain a drug reaction prediction result for the drug to be predicted based on the target representation.

In this embodiment, obtaining representations based on the target graph, since the target graph includes an entity graph and an interaction graph, which represent the internal topological information of entities and the correlation information between entities, respectively, this allows for predictions based on both internal and external information, thereby improving the accuracy of drug reaction prediction; additionally, obtaining the target representation based on the prompt identifier and the initial representation enables prompt learning on the representation, facilitating the effective transfer of pre-trained knowledge and further enhancing prediction accuracy.
In some embodiments, the multiple levels of entities include: a drug molecule and a target substructure of the drug molecule;
The entity graph includes: the molecular structure graph corresponding to the drug molecule, and the substructure graph corresponding to the target substructure;

The representation extraction module 602 is further configured to perform at least one of the following:
Using a pre-trained first target encoder to encode the molecular structure graph and the substructure graph to obtain an initial molecular representation;
Using a pre-trained second target encoder to encode the interaction graph to obtain an initial substructure representation;

The initial representation include: the initial molecular representation and the initial substructure representation.

In this embodiment, by using two target encoders, initial molecular representation and initial substructure representation can be obtained separately, which can leverage the excellent performance of the encoders to improve the accuracy of the initial representation, thereby enhancing prediction accuracy.
In some embodiments, the prompt identifier include: a molecular prompt identifier and a substructure prompt identifier;
The initial representation include: the initial molecular representation and the initial substructure representation;

The representation extraction module 602 is further configured to perform at least one of the following:
Obtaining a prompted molecular representation based on the initial molecular representation and the initial substructure representation corresponding to the initial molecular representation; and obtaining a target molecular representation based on the molecular prompt identifier and the prompted molecular representation;
Obtaining a prompted substructure representation based on the initial substructure representation and the initial molecular representation of a neighboring entity corresponding to the initial substructure representation; and obtaining a target substructure representation based on the substructure prompt identifier and the prompted substructure representation;

The target representation include: the target molecular representation and the target substructure representation.

In this embodiment, processing the initial representation based on the prompt identifier to obtain the target representation can use prompt learning to align downstream objectives with pre-training tasks, facilitating knowledge transfer and improving prediction accuracy.

In some embodiments, the representation extraction module 602 is further configured to:

Add the initial molecular representation with the initial substructure representation corresponding to the initial molecular representation to obtain the prompted molecular representation.

In some embodiments, the representation extraction module 602 is further configured to:

Add the initial substructure representation with the initial molecular representation of the neighboring entity corresponding to the initial molecular representation to obtain the prompted substructure representation.

In this embodiment, by adding the initial molecular representation and the initial substructure representation to obtain the prompted representation, information from both the drug molecule and substructure perspectives can be integrated, enhancing the expressive power of the prompted representation and thereby improving the accuracy of drug reaction prediction.

In some embodiments, the representation extraction module 602 is further configured to:

Use a pre-trained first parameterization network to parameterize the molecular prompt identifier to obtain a molecular prompt representation; concatenate the molecular prompt representation with the prompted molecular representation to obtain the target molecular representation.

In some embodiments, the representation extraction module 602 is further configured to:

Use a pre-trained second parameterization network to parameterize the substructure prompt identifier to obtain the substructure prompt representation; concatenate the substructure prompt representation with the prompted substructure representation to obtain the target substructure representation.

In this embodiment, by concatenating the prompt representation with the prompted representation to obtain the target representation, prompt information can be embedded in the target representation, facilitating better prediction and improving prediction performance.
In some embodiments, the target representation include: target molecular representation and target substructure representation;

The prediction module 604 is further configured to:
Use a pre-trained first classification network to classify the target molecular representation to obtain the first prediction result;
Use a pre-trained second classification network to classify the target substructure representation to obtain the second prediction result;
Obtain the drug reaction result based on the first prediction result and the second prediction result.

In this embodiment, obtaining the final prediction result based on the first and second prediction results allows for the integration of prediction information from both the drug molecule perspective and the drug substructure perspective, thereby improving the accuracy of the final prediction result.

In some embodiments, the prediction module 604 is further configured to:
Use a pre-trained linear projection layer to process the target molecular representation and the target substructure representation to obtain weights;

Based on these weights, perform a weighted summation on the first prediction result and the second prediction result to obtain the drug reaction result.

In this embodiment, by determining the weights and performing a weighted summation on the two prediction results based on these weights, it is possible to more accurately integrate the prediction information from both the drug molecule and the drug substructure, thereby improving the accuracy of the drug reaction prediction result.

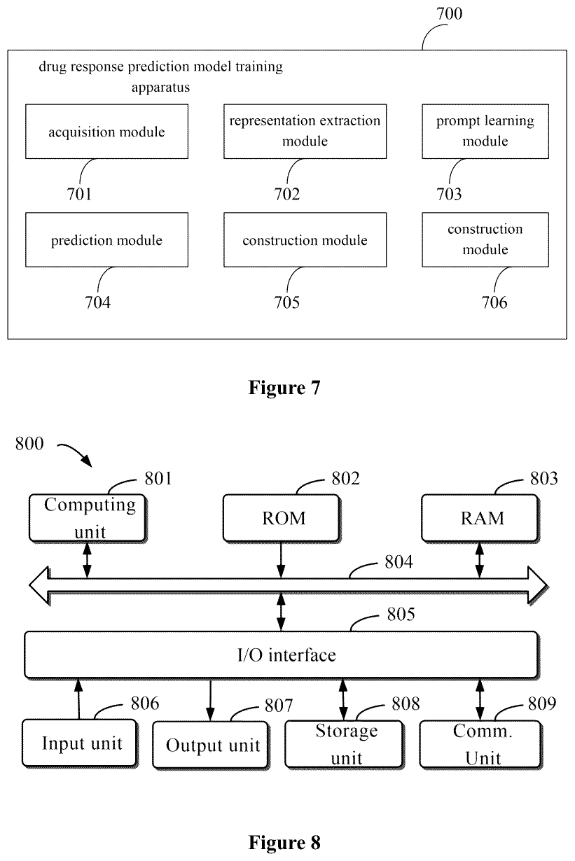

FIG. 7 is a schematic diagram according to a fifth embodiment of the present disclosure.

This embodiment provides a drug reaction prediction model training apparatus, where the drug reaction prediction model includes a representation extraction network, a prompt learning network, and a classification network. The apparatus 700 includes: an acquisition module 701, a representation extraction module 702, a prompt learning module 703, a prediction module 704, a construction module 705, and an adjustment module 706.

The acquisition module 701 is configured to obtain a target graph based on multiple levels of entities contained in a drug sample to be predicted, the target graph including an entity graph representing the topological information within the entities and an interaction graph representing the correlation information between the entities; the representation extraction module 702 is configured to use the representation extraction network to perform representation extraction processing on the target graph to obtain an initial representation; the prompt learning module 703 is configured to use the prompt learning network to obtain a target representation based on a learnable prompt identifier and the initial representation; the prediction module 704 is configured to use the classification network to obtain a predicted value of the drug reaction for the drug sample to be predicted based on the target representation; the construction module 705 is configured to construct a loss function based on the predicted value and the true value of the drug reaction for the drug sample to be predicted; the adjustment module 706 is configured to use the loss function to adjust the model parameters of the representation extraction network, the prompt learning network, and the classification network.

In this embodiment, obtaining representations based on the target graph, since the target graph includes entity graphs and interaction graphs, which represent the internal topological information of entities and the correlation information between entities, respectively, this allows for training based on both internal and external information, improving the model's effectiveness and thus enhancing the accuracy of drug reaction prediction; additionally, obtaining target representations based on prompt identifiers and initial representations enables prompt learning on the representations, facilitating the effective transfer of pre-trained knowledge and further enhancing the model's and prediction accuracy.
In some embodiments, the multiple levels of entities include: a drug molecule and a target substructure of the drug molecule;
The entity graph includes: a molecular structure graph corresponding to the drug molecule, and a substructure graph corresponding to the target substructure;
The representation extraction network includes: a first target encoder and a second target encoder;

The representation extraction module 702 is further configured to perform at least one of the following:
Using the first target encoder to encode the molecular structure graph and the substructure graph to obtain an initial molecular representation;
Using the second target encoder to encode the interaction graph to obtain an initial substructure representation;
The initial representation include: the initial molecular representation and the initial substructure representation.

In this embodiment, by using two target encoders, initial molecular representations and initial substructure representations can be obtained separately, which can leverage the excellent performance of the encoders to improve the accuracy of the initial representations, thereby enhancing prediction accuracy.
In some embodiments, the prompt identifier include: a molecular prompt identifier and a substructure prompt identifier;
The prompt learning network includes: a first parameterization network and a second parameterization network;

The representation extraction module 702 is further configured to perform at least one of the following:
Obtain a prompted molecular representation based on the initial molecular representation and the initial substructure representation corresponding to the initial molecular representation; use the first parameterization network to parameterize the molecular prompt identifier to obtain a molecular prompt representation; and obtain a target molecular representation based on the molecular prompt representation and the prompted molecular representation;
Obtain the prompted substructure representations based on the initial substructure representations and the initial molecular representation of the neighboring entity corresponding to the initial substructure representation; use the second parameterization network to parameterize the substructure prompt identifier to obtain substructure prompt representation; and obtain target substructure representation based on the substructure prompt representation and the prompted substructure representation;

The target representation include: the target molecular representation and the target substructure representation.

In this embodiment, processing the initial representations based on prompt identifiers to obtain target representations can use prompt learning to align downstream objectives with pre-training tasks, facilitating knowledge transfer and improving prediction accuracy.
In some embodiments, the target representation include: target molecular representation and target substructure representation;
The classification network includes: a first classification network and a second classification network;

The prediction module 704 is further configured to:
Use a first classification network to classify the target molecular representation to obtain a first prediction result;
Use a second classification network to classify the target substructure representation to obtain a second prediction result;

Obtain the drug reaction prediction value based on the first prediction result and the second prediction result.

In this embodiment, obtaining the final prediction result based on the first and second prediction results allows for the integration of prediction information from both the drug molecule perspective and the drug substructure perspective, thereby improving the accuracy of the final prediction result.
In some embodiments, the representation extraction network includes: a first target encoder and a second target encoder, both of which are pre-trained encoders; the apparatus further includes: a pre-training module;

The pre-training module is configured to:
Obtain a pre-trained molecular structure graph, a pre-trained substructure graph, and a pre-trained interaction graph based on a pre-training sample;
Use a first encoder to encode the pre-trained molecular structure graph and the pre-trained substructure graph to obtain the pre-trained molecular representation;
Use a second encoder to encode the pre-trained interaction graph to obtain the pre-trained substructure representation;
Construct a pre-training loss function based on the pre-trained molecular representation and the pre-trained substructure representation;
Adjust the model parameters of the first encoder and the second encoder based on the pre-training loss function until a pre-training termination condition is met;

Use the first encoder and the second encoder that meet the pre-training termination condition as the first target encoder and the second target encoder, respectively.

In this embodiment, the pre-training process can be used to obtain the first target encoder and the second target encoder, which allows for further fine-tuning on the pre-training basis to obtain the final drug reaction prediction model, thereby reducing workload and improving training efficiency and generalization.

In some embodiments, the pre-training module is further configured to:
Construct a molecular level loss function based on the pre-trained molecular representation;
Construct a substructure level loss function based on the pre-trained substructure representation;
Construct a contrastive loss function based on the pre-trained molecular representation and the pre-trained substructure representation;
Construct the pre-training loss function based on the molecular level loss function, the substructure level loss function, and the contrastive loss function.

In this embodiment, constructing the pre-training loss function based on the molecular level loss function, the substructure level loss function, and the contrastive loss function can combine multiple types of information to build the pre-training loss function, improving the pre-training effect and thus enhancing the model and prediction performance.

The drug reaction prediction model trained as described above can be used for the drug reaction process in any of the aforementioned embodiments, and due to the high accuracy of the drug reaction prediction model, the accuracy of drug reaction prediction can be improved.

It should be understood that, in the embodiments of the present disclosure, similar or identical content in different embodiments can be referred to each other.

It should be understood that, in the embodiments of the present disclosure, “first,” “second,” etc., are merely used for differentiation and do not indicate any order of importance, temporal sequence, etc.

The technical solutions of the present disclosure involve the collection, storage, use, processing, transmission, provision, and disclosure of user personal information, all of which comply with relevant laws and regulations and do not contravene public order and good customs.

According to embodiments of the present disclosure, the present disclosure also provides an electronic device, a non-transitory computer-readable storage medium, and a computer program product.

FIG. 8 illustrates a schematic block diagram of an example electronic device 800 that can be used to implement the embodiments of the present disclosure. Electronic device 800 is intended to represent various forms of digital computers, such as laptop computers, desktop computers, workstations, servers, blade servers, mainframe computers, and other suitable computers. Electronic device 800 can also represent various forms of mobile devices, such as personal digital assistants, cellular phones, smartphones, wearable devices, and other similar computing devices. The components shown herein, their connections and relationships, and their functions are merely examples, and are not intended to limit the implementation of the present disclosure as described and/or claimed herein.

As shown in FIG. 8, electronic device 800 includes a computing unit 801, which can perform various appropriate actions and processes according to computer programs stored in a read-only memory (ROM) 802 or loaded from a storage unit 808 into a random access memory (RAM) 803. Various programs and data required for the operation of electronic device 800 are also stored in RAM 803. The computing unit 801, ROM 802, and RAM 803 are interconnected via a bus 804. An input/output (I/O) interface 805 is also connected to the bus 804.

Multiple components of electronic device 800 are connected to the I/O interface 805, including: an input unit 806, such as a keyboard, mouse, etc.; an output unit 807, such as various types of displays, speakers, etc.; a storage unit 808, such as disks, optical discs, etc.; and a communication unit 809, such as a network card, modem, wireless communication transceiver, etc. The communication unit 809 allows electronic device 800 to exchange information/data with other devices via computer networks such as the Internet and/or various telecommunications networks.

The computing unit 801 can be various general and/or special-purpose processing components with processing and computing capabilities. Examples of computing unit 801 include, but are not limited to, a central processing unit (CPU), a graphics processing unit (GPU), various special-purpose artificial intelligence (AI) computing chips, various computing units running machine learning model algorithms, a digital signal processor (DSP), and any suitable processor, controller, microcontroller, etc. The computing unit 801 executes the various methods and processes described above, such as the drug reaction prediction method or the drug reaction prediction model training method. For example, in some embodiments, the drug reaction prediction method or the drug reaction prediction model training method can be implemented as a computer software program tangibly embodied in a machine-readable medium, such as storage unit 808. In some embodiments, portions or all of the computer program may be loaded and/or installed onto electronic device 800 via ROM 802 and/or communication unit 809. When the computer program is loaded into RAM 803 and executed by computing unit 801, one or more steps of the drug reaction prediction method or the drug reaction prediction model training method described above can be performed. Alternatively, in other embodiments, computing unit 801 may be configured to execute the drug reaction prediction method or the drug reaction prediction model training method by any other suitable means, such as through firmware.

Various implementations of the systems and techniques described above can be realized in digital electronic circuitry, integrated circuitry, field-programmable gate arrays (FPGA), application-specific integrated circuits (ASIC), application-specific standard products (ASSP), system-on-a-chip (SOC), complex programmable logic devices (CPLD), computer hardware, firmware, software, and/or combinations thereof. These various implementations may include: implementation in one or more computer programs that are executable and/or interpretable on a programmable system including at least one programmable processor, which may be special or general-purpose, configured to receive data and instructions from, and transfer data and instructions to, a storage system, at least one input device, and at least one output device.

Program code for implementing the methods disclosed herein may be written in any combination of one or more programming languages. This program code may be provided to a processor or controller of a general-purpose computer, special-purpose computer, or other programmable data processing apparatus, such that the program code, when executed by the processor or controller, causes the functions/operations specified in the flowcharts and/or block diagrams to be implemented. The program code may be entirely executed on the machine, partially executed on the machine, as a standalone software package, partially executed on the machine and partially executed on a remote machine, or entirely executed on the remote machine or server.

In the context of this disclosure, a machine-readable medium may be a tangible medium that can contain or store a program for use by or in connection with an instruction execution system, apparatus, or device. A machine-readable medium may be a machine-readable signal medium or a machine-readable storage medium. Machine-readable media may include, but are not limited to, an electronic, magnetic, optical, electromagnetic, infrared, or semiconductor system, apparatus, or device, or any suitable combination of the foregoing. More specific examples of a machine-readable storage medium would include an electrical connection based on one or more wires, a portable computer diskette, a hard disk, a random access memory (RAM), a read-only memory (ROM), an erasable programmable read-only memory (EPROM or flash memory), an optical fiber, a portable compact disc read-only memory (CD-ROM), an optical storage device, a magnetic storage device, or any suitable combination of the foregoing.

To provide interaction with a user, the systems and techniques described here can be implemented on a computer having a display device (e.g., a CRT (cathode ray tube) or LCD (liquid crystal display) monitor) for displaying information to the user; a keyboard; and a pointing device (e.g., a mouse or a trackball) by which the user can provide input to the computer. Other types of devices can also be used to provide for interaction with a user; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a backend component (e.g., as a data server), or that includes a middleware component (e.g., an application server), or that includes a frontend component (e.g., a user computer with a graphical user interface or a Web browser through which a user can interact with an implementation of the systems and techniques described here), or any combination of such backend, middleware, or frontend components. The components of the system can be interconnected by any form or medium of digital data communication (e.g., a communication network). Examples of communication networks include a LAN, a WAN, and the Internet.

The computing system can include clients and servers. A client and server are generally remote from each other and typically interact through a communication network. The relationship of client and server arises by virtue of computer programs running on the respective computers and having a client-server relationship to each other. A server can be a cloud server, also known as a cloud computing server or cloud host, which is a host product in the cloud computing service system, addressing the shortcomings of traditional physical hosts and VPS services (“Virtual Private Server,” or simply “VPS”) in terms of difficult management and weak business scalability. The server can also be a server in a distributed system, or a server combined with blockchain technology.

It should be understood that various forms of processes shown above can be used, with steps reordered, added, or removed. For example, the steps described in the present disclosure can be executed in parallel, sequentially, or in different orders, as long as the desired results of the technical solutions disclosed herein are achieved, and this is not restricted herein.

As used in the description herein and throughout the claims that follow, “a”, “an”, and “the” includes plural references unless the context clearly dictates otherwise.

The specific embodiments described above do not constitute a limitation on the scope of protection of the present disclosure. It should be understood by those skilled in the art that various modifications, combinations, sub-combinations, and substitutions may be made based on design requirements and other factors. Any modifications, equivalent replacements, and improvements made within the spirit and principle of the present disclosure are intended to be included within the scope of protection of the present disclosure.

What is claimed is:

1. A drug reaction prediction method, comprising:
obtaining a target graph based on multiple levels of entities contained in a drug to be predicted; wherein the target graph comprises an entity graph representing topological information within the entities and an interaction graph representing correlation information between the entities;
performing representation extraction processing on the target graph to obtain an initial representation;
obtaining a target representation based on a predetermined prompt identifier and the initial representation; and
obtaining a drug reaction prediction result for the drug to be predicted based on the target representation.

2. The method according to claim 1, wherein:
the multiple levels of entities comprise: a drug molecule and a target substructure of the drug molecule;
the entity graph comprises: a molecular structure graph corresponding to the drug molecule, and a substructure graph corresponding to the target substructure;
performing representation extraction processing on the target graph to obtain the initial representation comprises at least one of:
encoding the molecular structure graph and the substructure graph using a pre-trained first target encoder to obtain an initial molecular representation; and
encoding the interaction graph using a pre-trained second target encoder to obtain an initial substructure representation;
wherein the initial representation comprises the initial molecular representation and the initial substructure representation.

3. The method according to claim 1, wherein:
obtaining the target representation based on the predetermined prompt identifier and the initial representation comprises at least one of:
obtaining a prompted molecular representation based on an initial molecular representation and an initial substructure representation corresponding to the initial molecular representation; and obtaining a target molecular representation based on a molecular prompt identifier and the prompted molecular representation; and
obtaining a prompted substructure representation based on an initial substructure representation and an initial molecular representation of a neighboring entity corresponding to the initial molecular representation; and obtaining a target substructure representation based on a substructure prompt identifier and the prompted substructure representation;
wherein the initial representation comprises: the initial molecular representation and the initial substructure representation;
wherein the prompt identifier comprises: the molecular prompt identifier and the substructure prompt identifier; and
wherein the target representation comprises: the target molecular representation and the target substructure representation.

4. The method according to claim 3, wherein obtaining the prompted molecular representation based on the initial molecular representation and the initial substructure representation corresponding to the initial molecular representation comprises:
adding the initial molecular representation and the initial substructure representation corresponding to the initial molecular representation to obtain the prompted molecular representation.

5. The method according to claim 3, wherein obtaining the prompted substructure representation based on the initial substructure representation and the initial molecular representation of the neighboring entity corresponding to the initial substructure representation comprises:
adding the initial substructure representation and the initial molecular representation of the neighboring entity corresponding to the initial substructure representation to obtain the prompted substructure representation.

6. The method according to claim 3, wherein obtaining the target molecular representation based on the molecular prompt identifier and the prompted molecular representation comprises:
parameterizing the molecular prompt identifier using a pre-trained first parameterization network to obtain a molecular prompt representation; and
concatenating the molecular prompt representation with the prompted molecular representation to obtain the target molecular representation.

7. The method according to claim 3, wherein obtaining the target substructure representation based on the substructure prompt identifier and the prompted substructure representation comprises:
parameterizing the substructure prompt identifier using a pre-trained second parameterization network to obtain the substructure prompt representation; and
concatenating the substructure prompt representation and the prompted substructure representation to obtain the target substructure representation.

8. The method according to claim 1, wherein:
the target representation comprises: a target molecular representation and a target substructure representation;
wherein obtaining the drug reaction prediction result for the drug to be predicted based on the target representation comprises:
classifying the target molecular representation using a pre-trained first classification network to obtain a first prediction result;
classifying the target substructure representation using a pre-trained second classification network to obtain a second prediction result; and
obtaining the drug reaction prediction result based on the first prediction result and the second prediction result.

9. The method according to claim 8, wherein obtaining the drug reaction prediction result based on the first prediction result and the second prediction result comprises:
processing the target molecular representation and the target substructure representation using a pre-trained linear projection layer to obtain weights; and
performing weighted summation on the first prediction result and the second prediction result based on the weights to obtain the drug reaction prediction result.

10. The method according to claim 1, wherein the method is performed by a drug reaction prediction model comprising a representation extraction network, a prompt learning network, and a classification network;
wherein the method comprises:
using the representation extraction network to perform representation extraction processing on the target graph to obtain an initial representation;
using the prompt learning network to obtain a target representation based on a learnable prompt identifier and the initial representation;
using the classification network to obtain a predicted value of the drug reaction for the drug sample to be predicted based on the target representation;
constructing a loss function based on the predicted value and a true value of the drug reaction for the drug sample to be predicted; and
using the loss function to adjust the model parameters of the representation extraction network, the prompt learning network, and the classification network.

11. The method according to claim 10, wherein:
the representation extraction network comprises a first target encoder and a second target encoder, both of which are pre-trained encoders;
wherein the method further comprises:
obtaining a pre-trained molecular structure graph, a pre-trained substructure graph, and a pre-trained interaction graph based on a pre-training sample;
encoding the pre-trained molecular structure graph and the pre-trained substructure graph using a first encoder to obtain a pre-trained molecular representation;
encoding the pre-trained interaction graph using a second encoder to obtain a pre-trained substructure representation;
constructing a pre-training loss function based on the pre-trained molecular representation and the pre-trained substructure representation;
adjusting the model parameters of the first encoder and the second encoder based on the pre-training loss function until a pre-training termination condition is met; and
using the first encoder and the second encoder that meet the pre-training termination condition as the first target encoder and the second target encoder, respectively.

12. The method according to claim 11, wherein constructing the pre-training loss function based on the pre-trained molecular representation and the pre-trained substructure representation comprises:
constructing a molecular level loss function based on the pre-trained molecular representation;
constructing a substructure level loss function based on the pre-trained substructure representation;
constructing a contrastive loss function based on the pre-trained molecular representation and the pre-trained substructure representation; and
constructing the pre-training loss function based on the molecular level loss function, the substructure level loss function, and the contrastive loss function.

13. An electronic device, comprising:
at least one processor; and
a memory communicatively connected to the at least one processor; wherein
the memory stores instructions executable by the at least one processor, the instructions, when executed by the at least one processor, enabling the at least one processor to perform a drug reaction prediction method comprising:
obtaining a target graph based on multiple levels of entities contained in a drug to be predicted; wherein the target graph comprises an entity graph representing topological information within the entities and an interaction graph representing correlation information between the entities;
performing representation extraction processing on the target graph to obtain an initial representation;
obtaining a target representation based on a predetermined prompt identifier and the initial representation; and
obtaining a drug reaction prediction result for the drug to be predicted based on the target representation.

14. The electronic device according to claim 13, wherein:
the multiple levels of entities comprise: a drug molecule and a target substructure of the drug molecule;
the entity graph comprises: a molecular structure graph corresponding to the drug molecule, and a substructure graph corresponding to the target substructure;
performing representation extraction processing on the target graph to obtain the initial representation comprises at least one of:
encoding the molecular structure graph and the substructure graph using a pre-trained first target encoder to obtain an initial molecular representation; and
encoding the interaction graph using a pre-trained second target encoder to obtain an initial substructure representation;
wherein the initial representation comprises the initial molecular representation and the initial substructure representation.

15. The electronic device according to claim 13, wherein:
obtaining the target representation based on the predetermined prompt identifier and the initial representation comprises at least one of:
obtaining a prompted molecular representation based on an initial molecular representation and an initial substructure representation corresponding to the initial molecular representation; and obtaining a target molecular representation based on a molecular prompt identifier and the prompted molecular representation; and
obtaining a prompted substructure representation based on an initial substructure representation and an initial molecular representation of a neighboring entity corresponding to the initial molecular representation; and obtaining a target substructure representation based on a substructure prompt identifier and the prompted substructure representation;
wherein the initial representation comprises: the initial molecular representation and the initial substructure representation;
wherein the prompt identifier comprises: the molecular prompt identifier and the substructure prompt identifier; and
wherein the target representation comprises: the target molecular representation and the target substructure representation.

16. The electronic device according to claim 15, wherein obtaining the prompted molecular representation based on the initial molecular representation and the initial substructure representation corresponding to the initial molecular representation comprises:
adding the initial molecular representation and the initial substructure representation corresponding to the initial molecular representation to obtain the prompted molecular representation.

17. The electronic device according to claim 15, wherein obtaining the prompted substructure representation based on the initial substructure representation and the initial molecular representation of the neighboring entity corresponding to the initial substructure representation comprises:
adding the initial substructure representation and the initial molecular representation of the neighboring entity corresponding to the initial substructure representation to obtain the prompted substructure representation.

18. The electronic device according to claim 15, wherein obtaining the target molecular representation based on the molecular prompt identifier and the prompted molecular representation comprises:
parameterizing the molecular prompt identifier using a pre-trained first parameterization network to obtain a molecular prompt representation; and
concatenating the molecular prompt representation with the prompted molecular representation to obtain the target molecular representation.

19. The electronic device according to claim 15, wherein obtaining the target substructure representation based on the substructure prompt identifier and the prompted substructure representation comprises:
parameterizing the substructure prompt identifier using a pre-trained second parameterization network to obtain the substructure prompt representation; and
concatenating the substructure prompt representation and the prompted substructure representation to obtain the target substructure representation.

20. A non-transitory computer-readable storage medium storing computer instructions, wherein the computer instructions are configured to cause the computer to perform a drug reaction prediction method comprising:
obtaining a target graph based on multiple levels of entities contained in a drug to be predicted; wherein the target graph comprises an entity graph representing topological information within the entities and an interaction graph representing correlation information between the entities;
performing representation extraction processing on the target graph to obtain an initial representation;
obtaining a target representation based on a predetermined prompt identifier and the initial representation; and
obtaining a drug reaction prediction result for the drug to be predicted based on the target representation.
